(19) United States
(12) Patent Application Publication
(10) Pub. No.: US 2010/0010335 A1
(43) Pub. Date: Jan. 14, 2010

(54) METHOD AND APPARATUS FOR DIAGNOSING CANCER USING ELECTROMAGNETIC WAVE

(75) Inventors: Hyuk-Je Kim, Daejeon-si (KR); Jong-Moon Lee, Cheongju-si (KR); Youn-Ju Lee, Hwaseong-si (KR); Seong-Ho Son, Daejeon-si (KR); Soon-lk Jeon, Daejeon-si (KR)

Correspondence Address: RABIN & Berdo, PC, 1101 14TH STREET, NW, SUITE 500, WASHINGTON, DC, 20005, US

(73) Assignee: ELECTRONICS AND TELECOMMUNICATIONS RESEARCH INSTITUTE, Daejeon-si (KR)

(21) Appl. No.: 12/500,329

(22) Filed: Jul. 9, 2009

(30) Foreign Application Priority Data
Jul. 11, 2008 (KR) 10-2008-0067527

(51) Int. Cl. A61B 5/05

(52) U.S. Cl. 600/407

(57) ABSTRACT

A cancer diagnostic apparatus using electromagnetic waves includes: a plurality of antennas configured to transmit an electromagnetic wave signal to a human body and receive the electromagnetic wave signal from the human body; a switching unit configured to switch the antennas to a transmit mode or a receive mode; a signal generator configured to generate an electromagnetic wave signal to be transmitted to an antenna set to the transmit mode; a signal converter configured to convert an electromagnetic wave signal received from an antenna set to the receive mode into an intermediate frequency signal; and a controller including a switching controller to control the switching unit and a data processor to process a permittivity distribution image of the human body from the intermediate frequency signal.

### CROSS REFERENCE TO RELATED APPLICATION

This application claims the benefit under 35 U.S.C. § 119(a) of Korean Patent Application No. 10-2008-0067527, filed on Jul. 11, 2008, the disclosure of which is incorporated by reference in its entirety for all purposes.

### BACKGROUND

1. Field

The following description relates to a cancer diagnostic apparatus, and more particularly, to a cancer diagnostic apparatus using electromagnetic waves.

2. Description of the Related Art

There has been proposed breast cancer diagnostics using permittivity and conductivity differences in a human body through propagation characteristics of radio frequency (RF) signals with frequencies ranging from 500 to 3,000 MHz, for example. More specifically, traditional cancer diagnostic equipment includes a plurality of RF antennas, which are arranged circlewise, and signal converters, which combine RF signals received by the RF antennas with local oscillating (LO) signals and convert the combined signals to intermediate frequency (IF) signals.

Each signal converter receives the LO signal from an LO signal distributor. In this case, the RF signal is reversed through the LO signal path, thereby degrading channel isolation performance and thus lowering the accuracy in measurement of the RF signal.

Furthermore, each signal converter includes RF components. In this case, it is difficult to match RF signal measurement characteristics, such as gains and noise figures, of the respective RF components in measurement frequency bands. As a result, the accuracy in measurement of the RF signal in the diagnostic equipment may be decreased.

### SUMMARY

The present invention is directed to provide a cancer diagnostic apparatus using electromagnetic waves capable of preventing RF signals from flowing backward to a local oscillating (LO) power distributor for LO signal distribution.

The present invention is further directed to provide a cancer diagnostic apparatus with an improved accuracy in measurement of RF signals.

In one general aspect, there is provided a cancer diagnostic apparatus using electromagnetic waves, including: a plurality of antennas configured to transmit an electromagnetic wave signal to a human body and receive the electromagnetic wave signal from the human body; a switching unit configured to switch the antennas to a transmit mode or a receive mode; a signal generator configured to generate an electromagnetic wave signal to be transmitted to an antenna set to the transmit mode; a signal converter configured to convert an electromagnetic wave signal received from an antenna set to the receive mode into an intermediate frequency signal; and a controller including a switching controller to control the switching unit and a data processor to process a permittivity distribution image of the human body from the intermediate frequency signal.

The switching unit may include: a plurality of mode switches connected to the respective antennas to switch the antennas to the transmit mode or the receive mode; a transmit channel switch, including a input terminal connected to the signal generator and a plurality of output terminals connected to the respective mode switches; and a receive channel switch, including a plurality of input terminals connected to the mode switches and a output terminal connected to the signal converter.

The switching controller may control the switching unit to set one of the mode switches to the transmit mode in sequence one by one with the remaining mode switches set to the receive mode, and control the receive channel switch to receive the electromagnetic wave signal from the mode switches set to the receive mode in sequence.

According to another aspect, there is provided a cancer diagnostic method using electromagnetic waves, including: setting one of antennas to a transmit mode and the remaining to a receive mode; transmitting an electromagnetic wave signal to a human body through the antenna set to the transmit mode; receiving an electromagnetic wave signal from the human body through the antennas set to the receive mode in sequence one by one; and generating a permittivity distribution image using the received electromagnetic wave signal.

Other features will become apparent to those skilled in the art from the following detailed description, which, taken in conjunction with the attached drawings, discloses exemplary embodiments of the invention.

### BRIEF DESCRIPTION OF THE DRAWINGS

FIG. 1 is a block diagram of a cancer diagnostic apparatus according to an exemplary embodiment of the present invention.

FIG. 2 illustrates a switching unit in a cancer diagnostic apparatus according to an exemplary embodiment of the present invention.

FIG. 3 is a flow chart of a cancer diagnostic method according to an exemplary embodiment of the present invention.

Elements, features, and structures are denoted by the same reference numerals throughout the drawings and the detailed description, and the size and proportions of some elements may be exaggerated in the drawings for clarity and convenience.

### DETAILED DESCRIPTION

The following detailed description is provided to assist the reader in gaining a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

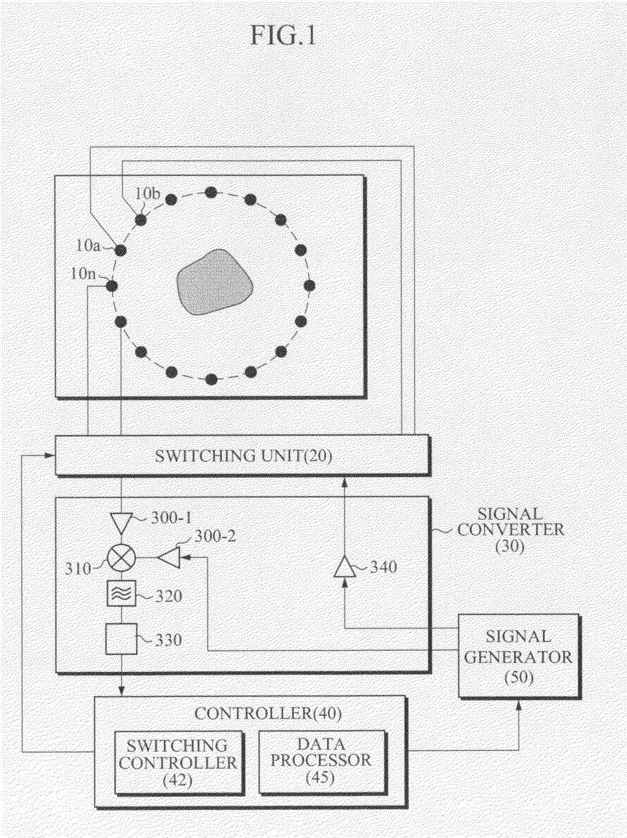

FIG. 1 is a block diagram of a cancer diagnostic apparatus according to an exemplary embodiment of the present invention.

The cancer diagnostic apparatus includes a plurality of antennas 10a, 10b, . . . , 10n, a switching unit 20, a signal converter 30, a controller 40 and a signal generator 50.

The signal generator 50 generates a radio frequency (RF) signal and a local oscillating (LO) signal.

The switching unit 20 switches each antenna to a receive mode or a transmit mode.

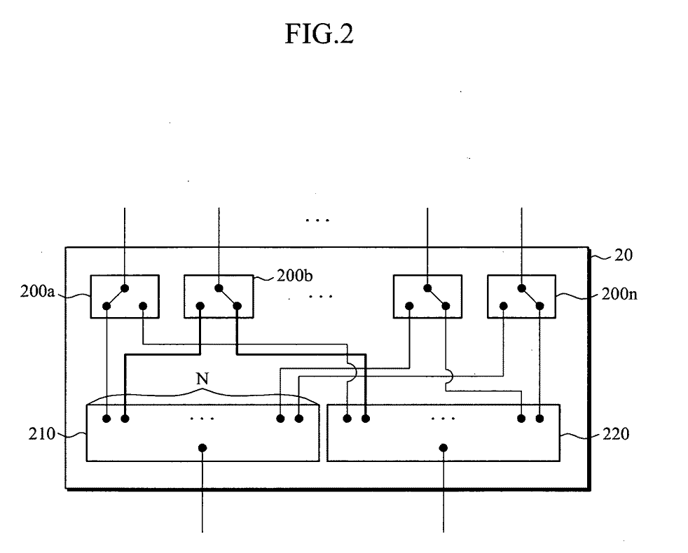

FIG. 2 illustrates the configuration of the switching unit 20 in the cancer diagnostic apparatus. The switching unit 20 includes N mode switches 200a, 200b, . . . , 200n, a transmit channel switch 210, and a receive channel switch 220. The respective N mode switches 200a, 200b, . . . , 200n are connected to N antennas to switch the respective N antennas to a transmit mode or a receive mode. The transmit channel switch 210 including an input terminal and N output terminals receives an electromagnetic wave signal through the input terminal and outputs it through its output terminal connected to a mode switch set to a transmit mode. The receive channel switch 220 including N input terminals and an output terminal receives an electromagnetic wave signal through one of its input terminals connected to a mode switch set to a receive mode and outputs it through its output terminal.

The signal converter 30 receives an RF signal from the signal generator 50 and sends the RF signal to the switching unit 20. Further, the signal converter 30 converts the RF signal from the switching unit 20 into an intermediate frequency (IF) signal and forwards it to a data processor 45.

More specifically, the signal converter 30 includes a first amplifier 300-1, a second amplifier 300-2, a mixer 310 and a low-pass filter 320. The first amplifier 300-1 amplifies an RF signal from the switching unit 20. The second amplifier 300-2 amplifies a local oscillating signal from the signal generator 50. The mixer 310 mixes the RF signal amplified by the first amplifier 300-1 and the local oscillating signal amplified by the second amplifier 300-2. The low-pass filter (LPF) 320 filters the mixed signal and outputs it to the data processor 45.

In the current example, the first and second amplifiers 300-1 and 300-2 each are a low noise amplifier (LNA).

The mixer 310 mixes the RF signal amplified by the first amplifier 300-1 and the local oscillating signal amplified by the second amplifier 300-2 to convert the RF signal to an IF signal.

The signal converter 30 further includes an analog-to-digital (A/D) converter 330 to convert the signal from the LPF 320 into a digital signal and send it to the data processor 45.

The signal converter 30 further includes a power amplifier 340 to amplify the RF signal form the signal generator 50. The power amplifier 340 amplifies the RF signal from the signal generator 50 and sends the amplified RF signal to the transmit antennas through the transmit channel switch 210.

The controller 40 includes a switching controller 42 and a data processor 45.

The switching controller 42 controls the switching unit 20. In the current example, the switching controller 42 controls the switching unit 20 to set one of the antennas to a transmit mode and the remaining to a receive mode. The switching controller 42 further controls the receive channel switch 220 to receive RF signals from the antennas set to the receive mode in sequence. After receiving the RF signal from the antennas set to the receive mode in sequence, the switching controller 42 controls the transmit channel switch 210 to set one of the remaining antennas to the transmit mode.

In a current example, the switching controller 42 controls the mode switch 200a of the first antenna 10a to be set to the transmit mode and the remaining switches 200b, . . . , 200n of the remaining antennas 10b, . . . , 10n to be set to the receive mode. The transmit channel switch 210 is controlled to output the RF signal to the terminal which is connected to the first antenna 10a. The receive channel switch 220 is controlled to output the RF signal which is sequentially received to its input terminals connected to the second antenna 10b, . . . , the n-th antenna 10n. After the RF signals are received through all of the receive mode antennas, the mode switches 200a, . . . , 200n are controlled such that the second antenna 10b is set to the transmit mode and the remaining antennas are set to the receive mode. The subsequent operations are the same as in the case where the first antenna 10a is set to the transmit mode.

More specifically, the switching controller 42 controls the switching unit 20 to operate an antenna in transmit mode and the other (n−1) antennas in receiver mode sequentially. The data processor 45 receives a digital signal from the signal converter 30 and generates a permittivity distribution image of and a conductivity distribution image of human body part.

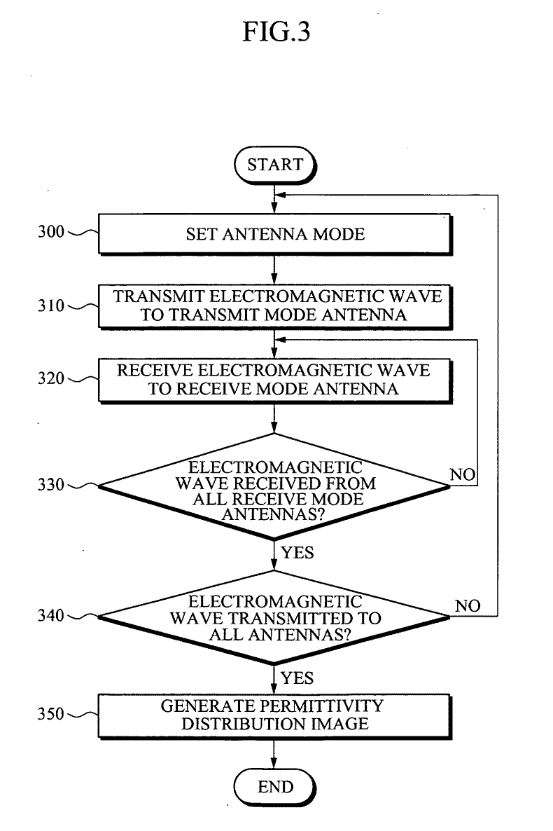

FIG. 3 is a flow chart of a cancer diagnostic method according to an exemplary embodiment of the present invention.

In operation 300, one of antennas is set to a transmit mode and the remaining are set to a receive mode. In operation 310, an electromagnetic wave signal is transmitted to a human body via the transmit mode antenna. In operation 320, the receive mode antennas receive an electromagnetic wave signal from the human body. In this case, the electromagnetic wave signal is received through the respective receive mode antennas in sequence. In operation 330, if the electromagnetic wave signal has been received through the respective receive mode antennas, another one of the antennas is set to the transmit mode. Similarly, in this case, antennas other than the antenna set to the transmit mode are set to the receive mode. An electromagnetic wave signal is received through the receive mode antennas in sequence. Until all of the antennas are operated in the transmit mode one by one, the electromagnetic wave signal is repeatedly transmitted and received while switching the antenna mode. If it is determined in operation 340 that all of the antennas have been operated in the transmit mode, in operation 350, the received electromagnetic wave signals are collected to generate the permittivity distribution image or the conductivity distribution image. The presence or absence of cancer in the human body can be detected from the image.

As apparent from the above description, by employing the single data processor and the channel switch instead of the LO power distributor, the breast cancer diagnostic apparatus using the electromagnetic waves can prevent a measurement error in channels and an interference between the channels. Accordingly, it is possible to obtain a more accurate measurement of the RF signal.

Furthermore, since the apparatus may be simply designed, it is possible to reduce the production cost.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced-or supplemented by other components or their equivalents. Accordingly, other implementations are within the scope of the following claims.

What is claimed is:

1. A cancer diagnostic apparatus using electromagnetic waves, comprising:
a plurality of antennas configured to transmit an electromagnetic wave signal to a human body and receive the electromagnetic wave signal from the human body;
a switching unit configured to switch the antennas to a transmit mode or a receive mode;
a signal generator configured to generate an electromagnetic wave signal to be transmitted to an antenna set to the transmit mode;
a signal converter configured to convert an electromagnetic wave signal received from an antenna set to the receive mode into an intermediate frequency signal; and
a controller including a switching controller to control the switching unit and a data processor to process a permittivity distribution image of the human body from the intermediate frequency signal.

2. The cancer diagnostic apparatus of claim 1, wherein the switching unit comprises:
a plurality of mode switches connected to the respective antennas to switch the antennas to the transmit mode or the receive mode;
a transmit channel switch, including a input terminal connected to the signal generator and a plurality of output terminals connected to the respective mode switches; and
a receive channel switch, including a plurality of input terminals connected to the mode switches and a output terminal connected to the signal converter.

3. The cancer diagnostic apparatus of claim 2, wherein the switching controller controls the switching unit to set one of the mode switches to the transmit mode in sequence one by one with the remaining mode switches set to the receive mode, and controls the receive channel switch to receive the electromagnetic wave signal from the mode switches set to the receive mode in sequence.

4. The cancer diagnostic apparatus of claim 1, wherein the signal converter comprises:
a first amplifier configured to amplify the electromagnetic wave signal from the switching unit;
a second amplifier configured to amplify a local oscillating signal;
a mixer configured to mix the amplified electromagnetic wave signal and the amplified local oscillating signal; and
a low-pass filter configured to filter the mixed signal and output the filtered signal to the data processor.

5. The cancer diagnostic apparatus of claim 4, wherein the signal converter further comprises a power amplifier configured to amplify electromagnetic wave signal from the signal generator and output the amplified electromagnetic wave signal through the switching unit.

6. A cancer diagnostic method using electromagnetic waves, comprising:
setting one of antennas to a transmit mode and the remaining to a receive mode;
transmitting an electromagnetic wave signal to a human body through the antenna set to the transmit mode;
receiving an electromagnetic wave signal from the human body through the antennas set to the receive mode in sequence one by one; and
generating a permittivity distribution image using the received electromagnetic wave signal.

7. The cancer diagnostic method of claim 6, further comprising, after receiving the electromagnetic wave signal,
if the electromagnetic wave signal is received from all of the receive mode antennas, switching the transmit mode antenna to the receive mode and setting one of the receive mode antennas to the transmit mode; and
repeating from transmitting the electromagnetic wave signal to switching the transmit mode antenna until the antennas are operated in the transmit mode in sequence.
